(19) United States
(12) Patent Application Publication
(10) Pub. No.: US 2012/0165677 A1
(43) Pub. Date: Jun. 28, 2012

(54) MEDICAL IMAGING SYSTEM AND MEDICAL IMAGING METHOD THEREOF

(75) Inventors: Pai-Chi LI, Taipei (TW); Wan-Ya Chen, Taipei (TW)

(73) Assignee: Pai-Chi Li, Taipei (TW)

(21) Appl. No.: 13/116,286

(22) Filed: May 26, 2011

(30) Foreign Application Priority Data
Dec. 24, 2010 (TW) 099145923

(51) Int. Cl. A61B 8/00

(52) U.S. Cl. 600/459

(57) ABSTRACT

A medical imaging system and a medical imaging method thereof are provided. The medical imaging method includes the following steps: acquiring an ultrasound image and a photoacoustic image; and overlapping the ultrasound image and the photoacoustic image to generate an overlapped image.

### BACKGROUND

1. Technical Field

The present invention generally relates to fields of medical imaging technologies, and particularly to a medical imaging system and a medical imaging method.

2. Description of the Related Art

So far, ultrasound imaging technology and photoacoustic imaging technology have been widely used in fields of medical testing. Both of the technologies have their advantages and disadvantages. Ultrasound images acquired by the ultrasonic imaging technology have higher spatial resolution, however, contrast of the ultrasound images is not quite good, makes testers not easily distinguish soft tissues and their nearby tiny blood vessels of similar acoustic resistance. And, the photoacoustic imaging technology can acquire photoacoustic images with better contrast; however, spatial resolution of the photoacoustic images is lower than that of the conventional ultrasound images.

To solve the above-mentioned problems, some testers simultaneously use an ultrasound imaging system and a photoacoustic imaging system to acquire ultrasound images and photoacoustic images, respectively. Then the ultrasound images and the photoacoustic images are paratactic displayed or are optionally played, in order to examine health conditions of patients. However, this method is not only inconvenient for testers, but also some misses may easily exist during testers matching the two kinds of images, thereby resulting in miscalculation of illness.

### BRIEF SUMMARY

Accordingly, the present invention is directed to a medical imaging system, in order to provide a convenient test method for testers, and overcome some existing bad misses by matching the two images and miscalculation of illness associated with the prior art.

The present invention is also directed to a medical imaging method applied to the medical imaging system.

Specifically, a medical imaging system in accordance with an embodiment of the present invention adapted to test an object under test is provided. The medical imaging system includes a broadband ultrasound probe, a laser transmission unit, an analog-digital converter, a digital-analog converter, a front-end processing circuit, and an image processing device. The analog-digital converter is connected to the broadband ultrasound probe. The digital-analog converter is connected to the broadband ultrasound probe and the laser transmission unit. The front-end processing circuit is connected to the analog-digital converter and the digital-analog converter. The front-end processing circuit controls the broadband ultrasound probe via the digital-analog converter in a first time zone, to output ultrasound to the object under test for generating a reflection signal of the ultrasound. The front-end processing circuit receives the reflection signal of the ultrasound detected by the broadband ultrasound probe, via the analog-digital converter in the first time zone. The front-end processing circuit controls the laser transmission unit via the digital-analog converter in a second time zone, to emit laser light to illuminate the object under test, for generating a photoacoustic signal. The front-end processing circuit receives the photoacoustic signal detected by the broadband ultrasound probe, via the analog-digital converter in the second time zone. The image processing device is connected to the front-end processing circuit. The image processing device establishes an ultrasound image and a photoacoustic image according to the reflection signal of the ultrasound and the photoacoustic signal acquired by the front-end processing circuit, and overlaps the ultrasound image and the photoacoustic image to generate an overlapped image.

In one embodiment of the present invention, the image processing device overlaps the photoacoustic image with the ultrasound image by means of chromatic, according to intensity of the photoacoustic signal.

In one embodiment of the present invention, the image processing device further gives different weights to the images corresponding to subbands of the photoacoustic signal, and combines these images to form the photoacoustic image, to enable the photoacoustic image to have higher contrast.

In one embodiment of the present invention, the image processing device includes a computer device. The computer device includes a graphics processing unit. The graphics processing unit is configured to overlap the ultrasound image and the photoacoustic image to generate the overlapped image.

In one embodiment of the present invention, the medical imaging system further includes a power amplifying unit. The power amplifying unit is connected between the broadband ultrasound probe and the digital-analog converter, and is connected between the broadband ultrasound probe and the analog-digital converter. The power amplifying unit amplifies signals outputted by the broadband ultrasound probe and the digital-analog converter.

In one embodiment of the present invention, the broadband ultrasound probe includes a first annular structure and a second annular structure. The first annular structure is set in the second annular structure. The first annular structure and the second annular structure are configured to be coaxial with each other. Thickness of the first annular structure is less than that of the second annular structure. A center frequency of the first annular structure is higher than that of the second annular structure. The first annular structure is configured to receive the reflection signal of the ultrasound, and the second annular structure is configured to receive the photoacoustic signal.

In one embodiment of the present invention, the broadband ultrasound probe further includes a perforation, so that the laser light passes through the perforation to illuminate the object under test.

In one embodiment of the present invention, the medical imaging system further includes a light guiding device. The light guiding device is set in the perforation for focusing the laser light, so that the object under test is illuminated by the focused laser light.

In one embodiment of the present invention, the light guiding device comprises a lens.

In one embodiment of the present invention, the light guiding device comprises an optical fiber. One terminal of the optical fiber is configured to receive the laser light emitted by the laser transmission unit.

In one embodiment of the present invention, the medical imaging system further includes a motor and a positioning control circuit. The motor is connected to the broadband ultrasound probe, to move the broadband ultrasound probe. The positioning control circuit controls the motor to move the broadband ultrasound probe.

A medical imaging method in accordance with another embodiment of the present invention is provided. The medical imaging method includes the following steps of: (1) acquiring an ultrasound image and a photoacoustic image; and (2) overlapping the ultrasound image and the photoacoustic image to generate an overlapped image.

In one embodiment of the present invention, the step of overlapping the ultrasound image and the photoacoustic image is: overlapping the photoacoustic image with the ultrasound image by means of chromatic, according to intensity of the photoacoustic signal.

In one embodiment of the present invention, the step of acquiring a photoacoustic signal includes: giving different weights to the images corresponding to subbands of the photoacoustic signal, and combining these images to form the photoacoustic image, to enable the photoacoustic image to have higher contrast.

The present invention provides a medical imaging system which can perform an ultrasound scanning and a laser scanning by means of time sharing to respectively acquire the ultrasound image and the photoacoustic image. And the medical imaging system can also overlap the acquired ultrasound image and the photoacoustic image to generate an overlapped image. Because the overlapped image simultaneously has a higher contrast than the photoacoustic image and a higher spatial resolution than the conventional ultrasound image, therefore, only the overlapped image is needed to be tested, testing procession can be easily performed, and some existing misses by matching two images can be overcome and miscalculation of illness can be largely reduced.

Other objectives, features and advantages of the present invention will be further understood from the further technological features disclosed by the embodiments of the present invention wherein there are shown and described preferred embodiments of this invention, simply by way of illustration of modes best suited to carry out the invention.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows a schematic diagram of a medical imaging system in accordance with an exemplary embodiment of the present invention.

FIG. 2 shows a schematic diagram of a medical imaging system in accordance with another exemplary embodiment of the present invention.

FIG. 3 shows a flowchart of a medical imaging method in accordance with an exemplary embodiment of the present invention.

### DETAILED DESCRIPTION

In the following detailed description of the preferred embodiments, reference is made to the accompanying drawings which form a part hereof, and in which are shown by way of illustration specific embodiments in which the invention may be practiced. It is to be understood that other embodiment may be utilized and structural changes may be made without departing from the scope of the present invention. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Accordingly, the descriptions will be regarded as illustrative in nature and not as restrictive.

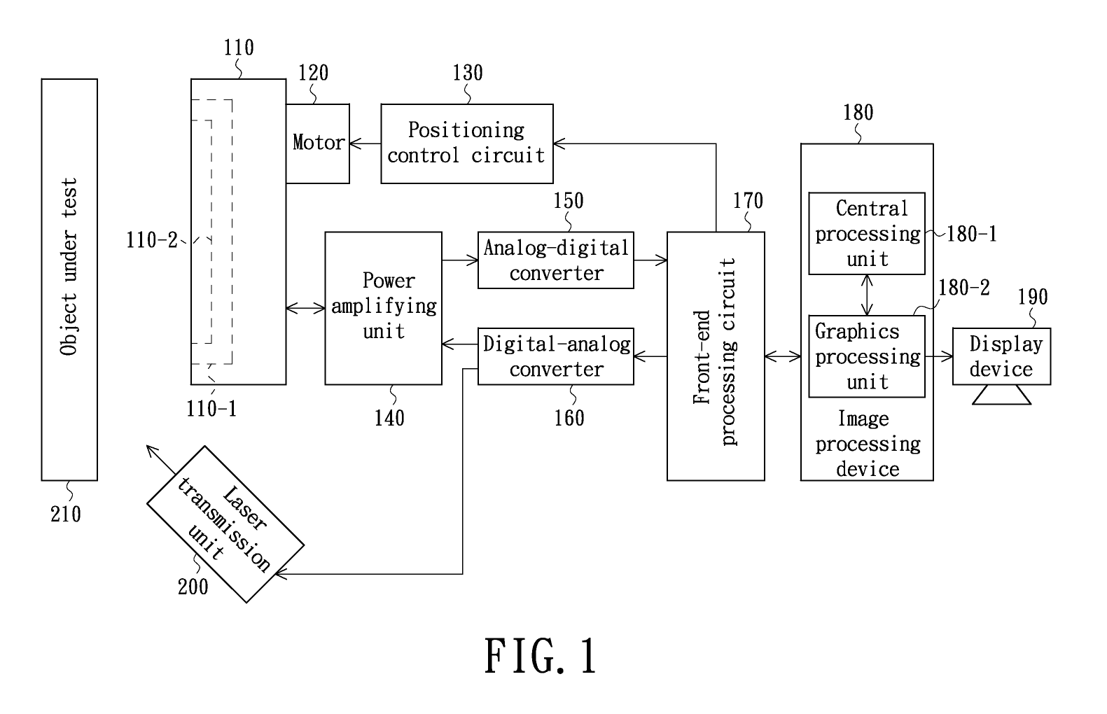

FIG. 1 shows a schematic diagram of a medical imaging system in accordance with an exemplary embodiment of the present invention. The medical imaging system is adapted to test an object under test 210. The medical imaging system includes a broadband ultrasound probe 110, a laser transmission unit 200, an analog-digital converter 150, a digital-analog converter 160, a front-end processing circuit 170, and an image processing device 180. The analog-digital converter 150 is connected to the broadband ultrasound probe 110. The digital-analog converter 160 is connected to the broadband ultrasound probe 110 and the laser transmission unit 200. The front-end processing circuit 170 is connected to the analog-digital converter 150 and the digital-analog converter 160. The image processing device 180 is connected to the front-end processing circuit 170.

The front-end processing circuit 170 is configured for controlling the broadband ultrasound probe 110 via the digital-analog converter 160 in a first time zone, to output ultrasound to the object under test 210 for obtaining a corresponding reflection signal from the object under test 210. The front-end processing circuit 170 is also configured for receiving reflection signal of the ultrasound detected by the broadband ultrasound probe 110, via the analog-digital converter 150 in the first time zone. In addition, the front-end processing circuit 170 is further configured for controlling the laser transmission unit 200 via the digital-analog converter 160 in a second time zone, to emit laser light to illuminate the object under test 210, thereby enabling the object under test 210 to generate a photoacoustic signal. The front-end processing circuit 170 is also configured to receive the photoacoustic signal detected by the broadband ultrasound probe 110, via the analog-digital converter 150 in the second time zone. In this embodiment, the front-end processing circuit 170 may be implemented by field programmable gate arrays (FPGAs). But it should be noted that, the first time zone and the second time zone are not overlapped with each other.

The image processing device 180 establishes an ultrasound image (that is, acquires an ultrasound frame) according to the reflection signal of the ultrasound acquired by the front-end processing circuit 170 in the first time zone, and also establishes a photoacoustic image (that is, acquires a photoacoustic frame) according to the photoacoustic signal acquired by the front-end processing circuit 170 in the second time zone. In addition, the image processing device 180 also overlaps the ultrasound image and the photoacoustic image to generate an overlapped image. For example, the image processing device 180 may overlap the photoacoustic image with the ultrasound image by means of chromatic, according to intensity of the photoacoustic signal, to generate the above-mentioned overlapped image. Preferably, the image processing device 180 can further give different weights to the images corresponding to subbands of the photoacoustic signal, and combine these images to form the photoacoustic image, to enable the photoacoustic image to have higher contrast. Thus, the overlapped image generated by overlapping ultrasound image and the photoacoustic image has a higher contrast. In addition, in order to achieve real-time display of the photoacoustic image, the laser pulse repetition frequency of the laser transmission unit 200 is preferably greater than kHz.

In addition, the image processing device 180 may be implemented by a computer device. The computer device includes a central processing unit 180-1 and a graphics processing unit 180-2. The graphics processing unit 180-2 establishes an ultrasound image according to the reflection signal of the ultrasound acquired by the front-end processing circuit 170 in the first time zone, and also establishes a photoacoustic image according to the photoacoustic signal acquired by the front-end processing circuit 170 in the second time zone. In addition, the graphics processing unit 180-2 also overlaps the ultrasound image and the photoacoustic image to generate an overlapped image. The central processing unit 180-1 executes relevant application programs of image processing, so that users can set or alter an image processing method of the graphics processing unit 180-2 via the relevant application programs.

The medical imaging system can further include a display device 190. The display device 190 is connected to the graphics processing unit 180-2. Thus, the display device 190 can display the ultrasound image, the photoacoustic image, and the overlapped image acquired by the graphics processing unit 180-2. Of course, the display device 190 can also display the above-mentioned scenes of the relevant application programs. In addition, the medical imaging system can further include a power amplifying unit 140. The power amplifying unit 140 is connected between the broadband ultrasound probe 110 and the digital-analog converter 160, to conveniently amplify the signals outputted by the broadband ultrasound probe 110 and the digital-analog converter 160.

The broadband ultrasound probe 110 includes annular structures 110-1 and 110-2. The annular structure 110-2 is set in the annular structure 110-1, and the annular structure 110-2 and the annular structure 110-1 are appropriately configured to be coaxial with each other. In addition, thickness of the annular structure 110-2 is less than that of the annular structure 110-1, and center frequency of the annular structure 110-2 is higher than that of the annular structure 110-1. The annular structure 110-2 is configured to receive reflection signal of the ultrasound, and the annular structure 110-1 is configured to receive the photoacoustic signal.

The broadband ultrasound probe 110 can further include a perforation (not shown in FIG. 1), so that the laser light emitted by the laser transmission unit 200 can pass through the perforation to illuminate the object under test 210. And position of the perforation can be set in the axis of the annular structures 110-1 and 110-2. In addition, the medical imaging system can further include a motor 120 and a positioning control circuit 130. The motor 120 is connected to the broadband ultrasound probe 110, in order to move the broadband ultrasound probe 110. And the positioning control circuit 130 controls the motor 120 to move the broadband ultrasound probe 110. The motor may be a sound coil motor, and the positioning control circuit 130 may be a digital signal processor (DSP).

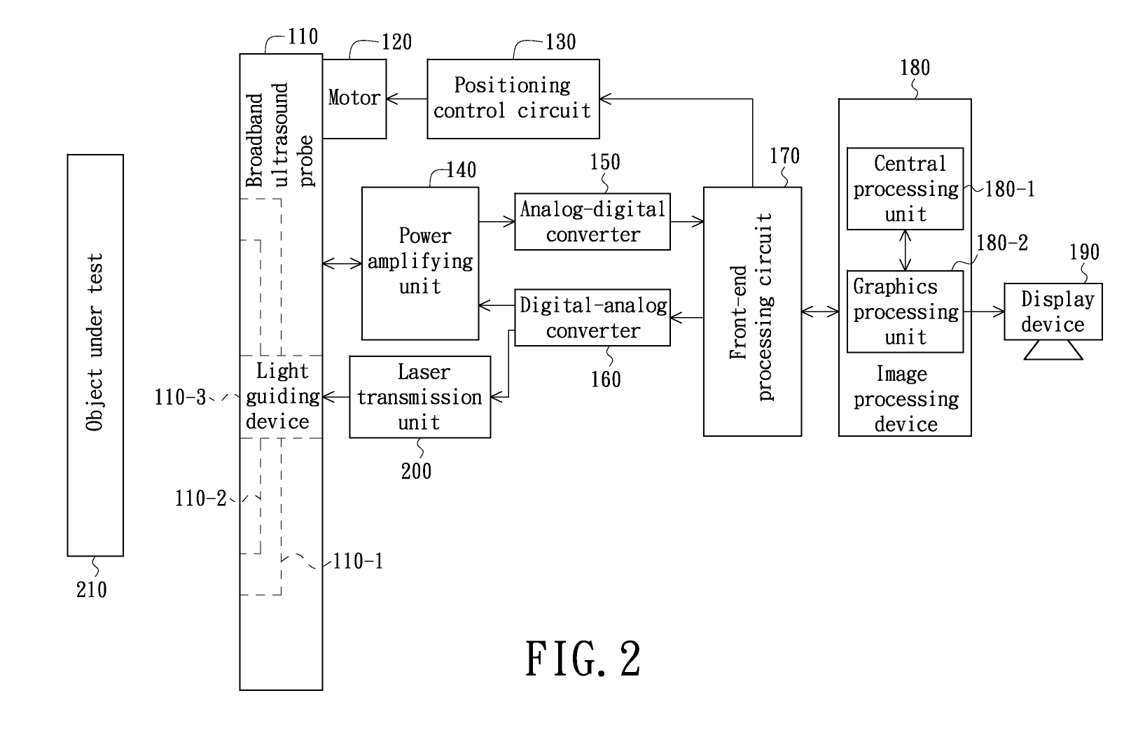

Preferably, the medical imaging system can further includes a light guiding device as shown in FIG. 2. FIG. 2 shows a schematic diagram of a medical imaging system in accordance with another exemplary embodiment of the present invention. In FIG. 2, the object marked with number 110-3 is the light guiding device. The light guiding device 110-3 is set in the perforation (not labeled), for focusing the laser light emitted by the laser transmission unit 200, so that the object under test 210 can be illuminated by the focused laser light. The light guiding device 110-3 may be a lens or an optical fiber. When the light guiding device 110-3 is an optical fiber, one terminal of the optical fiber is configured to receive the laser light emitted by the laser transmission unit 200.

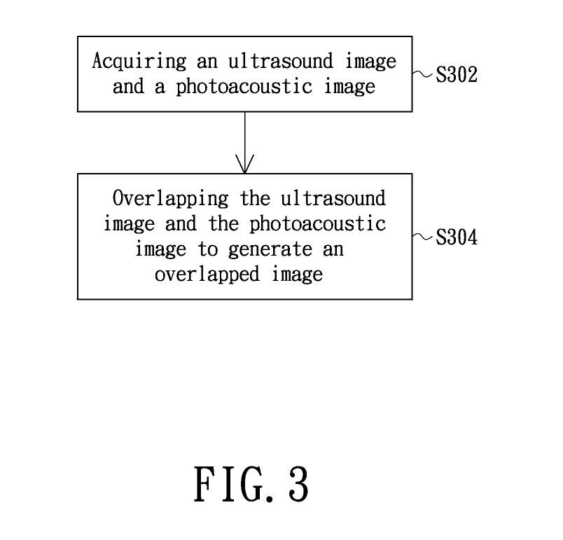

From the above illustration, it can be concluded that some basic operation procedures of the medical imaging system of the present invention as shown in FIG. 3. FIG. 3 shows a flowchart of a medical imaging method in accordance with an exemplary embodiment of the present invention. Referring to FIG. 3, the medical imaging method includes the following steps of: acquiring an ultrasound image and a photoacoustic image (as shown in step S302); overlapping the ultrasound image and the photoacoustic image to generate an overlapped image (as shown in step S304).

In summary, the present invention provides a medical imaging system which can perform an ultrasound scanning and a laser scanning by means of time sharing to respectively acquire the ultrasound image and the photoacoustic image. The medical imaging system can also overlap the acquired ultrasound image and the photoacoustic image to generate an overlapped image. Because the overlapped image simultaneously has a higher contrast than the photoacoustic image and a higher spatial resolution than the conventional ultrasound images, therefore, only the overlapped image is needed to be tested, testing procession can be easily performed, and some existing misses by matching two images can be overcome and miscalculation of illness can be largely reduced.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A medical imaging system adapted to test an object under test, the medical imaging system comprising:
a broadband ultrasound probe;
a laser transmission unit;
an analog-digital converter, connected to the broadband ultrasound probe;
a digital-analog converter, connected to the broadband ultrasound probe and the laser transmission unit;
a front-end processing circuit, connected to the analog-digital converter and the digital-analog converter, to control the broadband ultrasound probe via the digital-analog converter in a first time zone, to output ultrasound to the object under test for generating a reflection signal of the ultrasound, receive the reflection signal of the ultrasound detected by the broadband ultrasound probe, via the analog-digital converter in the first time zone, control the laser transmission unit via the digital-analog converter in a second time zone, to emit laser light to illuminate the object under test, for generating a photoacoustic signal, receive the photoacoustic signal detected by the broadband ultrasound probe via the analog-digital converter in the second time zone; and
an image processing device, connected to the front-end processing circuit, to establish an ultrasound image and a photoacoustic image according to the reflection signal of the ultrasound and the photoacoustic signal acquired by the front-end processing circuit, and overlap the ultrasound image and the photoacoustic image to generate an overlapped image.

2. The medical imaging system as claimed in claim 1, wherein the image processing device overlaps the photoacoustic image with the ultrasound image by means of chromatic, according to intensity of the photoacoustic signal.

3. The medical imaging system as claimed in claim 1, wherein the image processing device further gives different weights to the images corresponding to subbands of the photoacoustic signal, and combines these images to form the photoacoustic image, to enable the photoacoustic image to have higher contrast.

4. The medical imaging system as claimed in claim 1, wherein the image processing device comprises a computer device, the computer device comprises a graphics processing unit, and the graphics processing unit overlaps the ultrasound image and the photoacoustic image to generate the overlapped image.

5. The medical imaging system as claimed in claim 1, further comprising:
a power amplifying unit, connected between the broadband ultrasound probe and the digital-analog converter, and connected between the broadband ultrasound probe and the analog-digital converter to amplify signals outputted by the broadband ultrasound probe and the digital-analog converter.

6. The medical imaging system as claimed in claim 1, wherein the broadband ultrasound probe comprises a first annular structure and a second annular structure, the first annular structure is set in the second annular structure, the first annular structure and the second annular structure are appropriately configured to be coaxial with each other, thickness of the first annular structure is less than that of the second annular structure, a center frequency of the first annular structure is higher than that of the second annular structure, the first annular structure receives the reflection signal of the ultrasound, and the second annular structure receives the photoacoustic signal.

7. The medical imaging system as claimed in claim 1, wherein the broadband ultrasound probe further comprises a perforation, and the laser light passes through the perforation to illuminate the object under test.

8. The medical imaging system as claimed in claim 7, further comprising a light guiding device, wherein the light guiding device is set in the perforation for focusing the laser light, and so that the object under test is illuminated by the focused laser light.

9. The medical imaging system as claimed in claim 8, wherein the light guiding device comprises a lens.

10. The medical imaging system as claimed in claim 8, wherein the light guiding device comprises an optical fiber, and one terminal of the optical fiber receives the laser light emitted by the laser transmission unit.

11. The medical imaging system as claimed in claim 10, further comprising:
a motor, connected to the broadband ultrasound probe, to move the broadband ultrasound probe; and
a positioning control circuit, to control the motor to move the broadband ultrasound probe.

12. A medical imaging method comprising:
acquiring an ultrasound image and a photoacoustic image; and
overlapping the ultrasound image and the photoacoustic image to generate an overlapped image.

13. The medical imaging method as claimed in claim 12, wherein the step of overlapping the ultrasound image and the photoacoustic image comprising:
overlapping the photoacoustic image with the ultrasound image by means of chromatic, according to intensity of the photoacoustic signal.

14. The medical imaging method as claimed in claim 12, wherein the step of acquiring a photoacoustic signal comprising:
giving different weights to the images corresponding to subbands of the photoacoustic signal, and combining these images to form the photoacoustic image, to enable the photoacoustic image to have higher contrast.
